(19) United States
(12) Patent Application Publication
(10) Pub. No.: US 2026/0014373 A1
(43) Pub. Date: Jan. 15, 2026

(54) DEVICE FOR TREATMENT OF INCONTINENCE

(71) Applicant: FEMME MEDICAL LTD., Yehud-Monosson (IL)

(72) Inventors: Adrian Paz, Petah Tikva (IL); Yehuda Bachar, Yehud-Monosson (IL)

(73) Assignee: FEMME MEDICAL LTD., Yehud-Monosson (IL)

(21) Appl. No.: 19/125,321

(22) PCT Filed: Oct. 25, 2023
(86) PCT No.: PCT/IL2023/051105

§ 371 Date: Apr. 29, 2025

Related U.S. Application Data
(63) Continuation of application No. PCT/IL2023/051105, filed on Oct. 25, 2023.
(60) Provisional application No. 63/422,055, filed on Nov. 3, 2022.

(51) Int. Cl. A61N 1/36; A61N 1/05; A61N 1/372

(52) U.S. Cl. CPC A61N 1/36007; A61N 1/0512; A61N 1/0524; A61N 1/37217

(57) ABSTRACT

The present invention relates to a device for the prevention and/or treatment of fecal and/or urinary incontinence. The device may fit into a vagina. The device may include a pressure sensor and electrodes. The device may include a lateral projection on the device and/or a skin of the device. In response to intra-abdominal pressure the device may apply electrostimulation with the electrodes, for example to a vaginal mucosa.

### RELATED APPLICATION/S

This application claims the benefit of priority under 35 USC § 119(e) of U.S. Provisional Patent Application No. 63/422,055 filed 3 Nov. 2022, the contents of which are incorporated herein by reference in their entirety.

### FIELD AND BACKGROUND OF THE INVENTION

The present invention in some embodiments thereof relates to a device for the prevention and/or treatment of incontinence, and more particularly, but not exclusively, in women.

Accidental loss or leaking of urine, called urinary incontinence (UI), is one of the most common bladder control problems thought to affect millions of people. Urinary incontinence may be caused by loss of muscle tonicity, e.g., due to age or infirmity, damage during childbirth, increased abdominal pressure due to pregnancy or obesity, damage to the bladder or nearby area during surgery, neurological conditions that affect the brain and spinal cord, such as Parkinson's disease or multiple sclerosis, conditions affecting the lower urinary tract (urethra and bladder)—such as urinary tract infections (UTIs) or tumors in the bladder, certain medicines such as angiotensin converting enzyme (ACE) inhibitors, diuretics, some antidepressants, hormone replacement therapy (HRT), sedatives, etc.

There are various types of urinary incontinence: for example, urgency, stress, functional and overflow incontinence:
Stress incontinence—urine leaks out at times when the bladder is under pressure, e.g., when coughing, sneezing or laughing. This can be caused by a lack of support of the bladder neck and/or due to weakness of pelvic floor. In this condition, when intra-abdominal pressure increases the unsupported bladder neck cannot hold back the urine.
Urgency incontinence—intense urge to pee, or soon afterwards can result in urine leaks. This may be caused by involuntary detrusor muscle contraction causing a rise in intravesical (bladder) pressure, such a condition is known as detrusor overactivity.
Overflow incontinence (chronic urinary retention)—this occurs when a person is unable to fully empty their bladder, which can cause frequent leaking.
Total incontinence—when the bladder cannot store any urine at all, which causes constant urine passing or frequent leaking.

In many of cases, both stress and urgency urinary incontinence are present, with either type being predominant, known as mixed urinary incontinence.

In addition, bowel incontinence, or fecal incontinence, occurs when a person has problems controlling their bowels. Fecal incontinence may be caused by severe or long-lasting constipation or diarrhea, irritable bowel syndrome (IBS), inflammatory bowel disease, such as Crohn's disease, severe piles (hemorrhoids), childbirth or surgery damaging the muscles or nerves used to control the bowels, conditions that can affect the nerves, such as diabetes, a stroke or spina bifida, etc.

Urinary incontinence and fecal incontinence can occur in the same individual.

Current treatment methods for managing urinary and/or fecal incontinence include behavioral therapies, pelvic floor exercises, medications, nerve stimulation and surgery.

Therefore, there is a need for a means of preventing and/or treating urinary incontinence and/or fecal incontinence.

### SUMMARY OF THE INVENTION

According to an aspect of some embodiments of the invention, there is provided an electrostimulation device for stimulating the pelvic floor including: an electrode configured for intimate contact with vaginal or rectal mucosa; a sensor for detecting urinary related activity; and a processor configured to receive signals from the pressure sensor, and in response to detection of increasing intra-abdominal pressure by the pressure sensor, induce electrostimulation of a pelvic floor by the electrode.

According to some embodiments of the invention, the urinary related activity includes at least one of intra-abdominal pressure, pelvic floor contraction, and electromyographic activity of the pelvic floor.

According to some embodiments of the invention, the sensor includes a pressure sensor.

According to some embodiments of the invention, the processor and electrode are configured to respond to the sensor sensing increased activity of pelvic floor muscles by stimulating the pelvic floor muscles with positive feedback.

According to some embodiments of the invention, the positive feedback suppresses a voiding reflex.

According to some embodiments of the invention, the length of the device is adaptable to fit the length of a vagina.

According to some embodiments of the invention, at least one of a diameter and a lateral protrusion from the device is adaptable to fit the width of a vagina.

According to some embodiments of the invention, the device further includes a rim.

According to some embodiments of the invention, the device where the rim is flexible.

According to some embodiments of the invention, the flexibility of the rim facilitates insertion or removal of the device.

According to some embodiments of the invention, the device is configured to strengthen the pelvic floor and treat mild to moderate pelvic floor prolapse.

According to some embodiments of the invention, the device further includes a wireless communication module for sending data to an external computer and receiving signals from the external computer to adjust stimulation parameters.

According to some embodiments of the invention, the device further includes: a body is configured to support a vagina wall at a level of a bladder neck above a pubis.

According to some embodiments of the invention, the device further includes: an elastic body which is reversibly deformed by an increase in the intra-abdominal pressure.

According to some embodiments of the invention, the reversible deformation is a reversible reduction of an anteroposterior diameter of the body by at least 5% when the intra-abdominal pressure increases by at least 1/10 bar.

According to some embodiments of the invention, the body has an axially compressible region.

According to some embodiments of the invention, the body includes a lateral projection.

According to some embodiments of the invention, the lateral projection includes a rim around the body of the device.

According to some embodiments of the invention, the device further includes a lateral projection configured to support a bladder neck.

According to some embodiments of the invention, the device further includes a removal thread hanging caudally from the device and wherein the removal thread includes an antenna for wireless communication.

According to some embodiments of the invention, the device further includes a wireless communication module.

According to some embodiments of the invention, sensors recordings, stimulation parameters and timing are transmitted wirelessly to an external computing device, wherein the external computing device includes a user interface for controlling the device.

According to some embodiments of the invention, the pressure sensor is selected from a group consisting of strain gauge, piezoelectric crystal, piezoelectric film, balloon and any combination thereof.

According to some embodiments of the invention, the device further includes one or more additional sensors, wherein the one or more additional sensors are selected from a group consisting of: accelerometer, position, tilt, photoplethysmography, bioimpedance, ultrasonographic electromyography, and any combination thereof.

According to some embodiments of the invention, the processor is configured to administer the electrostimulation in a manner that strengthens pelvic floor muscles.

According to some embodiments of the invention, the processor is configured to administer the electrostimulation in a manner that produces neuromodulation.

According to some embodiments of the invention, the neuromodulation includes reflex contraction of the pelvic floor on increase in intra-abdominal pressure.

According to some embodiments of the invention, the neuromodulation includes inhibition of a desire to urinate or to defecate.

According to some embodiments of the invention, the electrostimulation causes neuromodulation which initiates voiding or defecation.

According to an aspect of some embodiments of the invention, there is provided a method for non-invasive electrostimulation of pelvic floor muscles including: sensing an increase in intra-abdominal pressure; electrostimulating the pelvic floor muscles by at least one electrode in contact with a vaginal or rectal mucosa in response to the sensing to contract the pelvic floor muscles in response to the electrostimulation.

According to some embodiments of the invention, the method further includes Repeating the sensing and electrostimulation to create a reflex to automatically contract the pelvic floor on increase of intra-abdominal pressure.

According to some embodiments of the invention, the electrostimulating reduces at least one of frequency and urgency of urination.

According to some embodiments of the invention, the method further includes Repeating the sensing and electrostimulation to strengthen pelvic floor muscles.

According to some embodiments of the invention, the method further includes controlling the electrostimulation by a processor is connected to at least one pressure sensor and at least one electrode.

According to some embodiments of the invention, the processor further includes an artificial intelligence (AI) module.

According to some embodiments of the invention, the method further includes determining with the AI module when there is a high chance of an imminent urine or fecal leak.

According to some embodiments of the invention, the method further includes instructing the at least one electrode to provide electrostimulation is response to the determining.

According to an aspect of some embodiments of the invention, there is provided a system for electrostimulation of pelvic floor muscles including: an insertable device for insertion into a vagina including an electrode configured for intimate contact with vaginal or rectal mucosa, a sensor for detecting urinary related activity and a wireless communication module, a processor configured to receive signals from the pressure sensor, and in response to detection of increasing intra-abdominal pressure by the pressure sensor, induce electrostimulation of the pelvic floor by the electrode; and a user interface linked wirelessly to the processor, wherein the user interface is an application on an external computer.

According to some embodiments of the invention, the urinary related activity includes at least one of intra-abdominal pressure, pelvic floor contraction, and electromyographic activity of the pelvic floor.

According to some embodiments of the invention, the wireless communication module is configured for sending data to the external computer and receiving signals from the external computer to adjust stimulation parameters.

According to some embodiments of the invention, the external computer includes a user interface configured to collect feedback from a user regarding at least one of occurrence, severity of incontinence episodes and context of incontinence episodes.

According to some embodiments of the invention, the external computer is configured to use these data in order to adjust the stimulation parameters.

According to some embodiments of the invention, the external computer is configured to present to a user data on at least one of pelvic floor strength, improvement with time and improvement regarding incontinence treatment.

According to some embodiments of the invention, the external computer is configured to use data from multiple users is for the adjusting.

According to some embodiments of the invention, the external computer is configured to use the data from multiple users for training a machine learning routine.

According to some embodiments of the invention, includes at least one of a vector machine and reinforcement learning.

According to some embodiments of the invention, the user interface is configured to facilitate wireless activation, control, and/or programing of one or more parameters of the insertable device.

According to some embodiments of the invention, the one or more parameters are selected from the group consisting of: cutoff values for the pressure sensor signals that induce electrostimulation, voltage, current intensity, shape and duration of stimulation pulses, duty cycle, modulation, session duration, patterns, and any combination thereof.

According to some embodiments of the invention, the external computer is a cellular phone, tablet, phablet, laptop, desktop, smartwatch, smart glasses, or VR device.

According to some embodiments of the invention, the external computer is configured to analyze, store, and present to user cumulative electrostimulation data, cumulative sensor data, changes, and trends in the data in a graphical manner.

### DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

### IN THE DRAWINGS

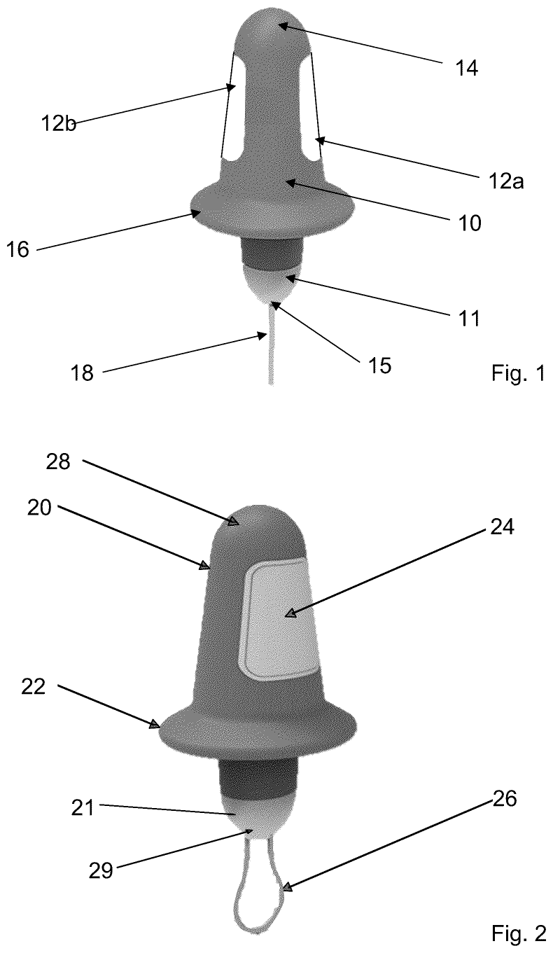

FIG. 1: A schematic illustration of a side view of an intravaginal device, in accordance with some embodiments.

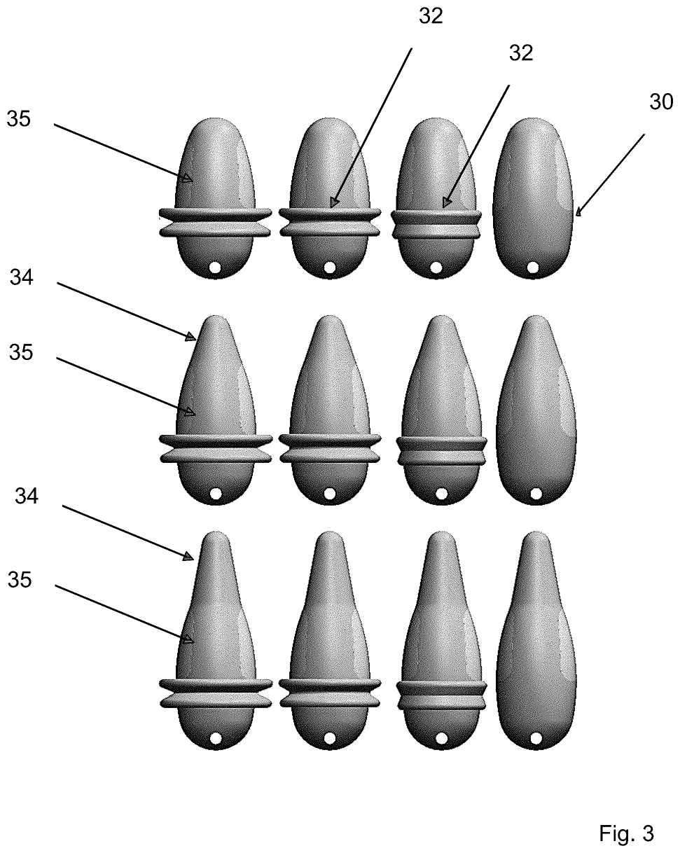

FIG. 2: A schematic illustration of a side view of an intravaginal device, in accordance with some embodiments.

FIG. 3: Side views of various intravaginal device, in accordance with some embodiments.

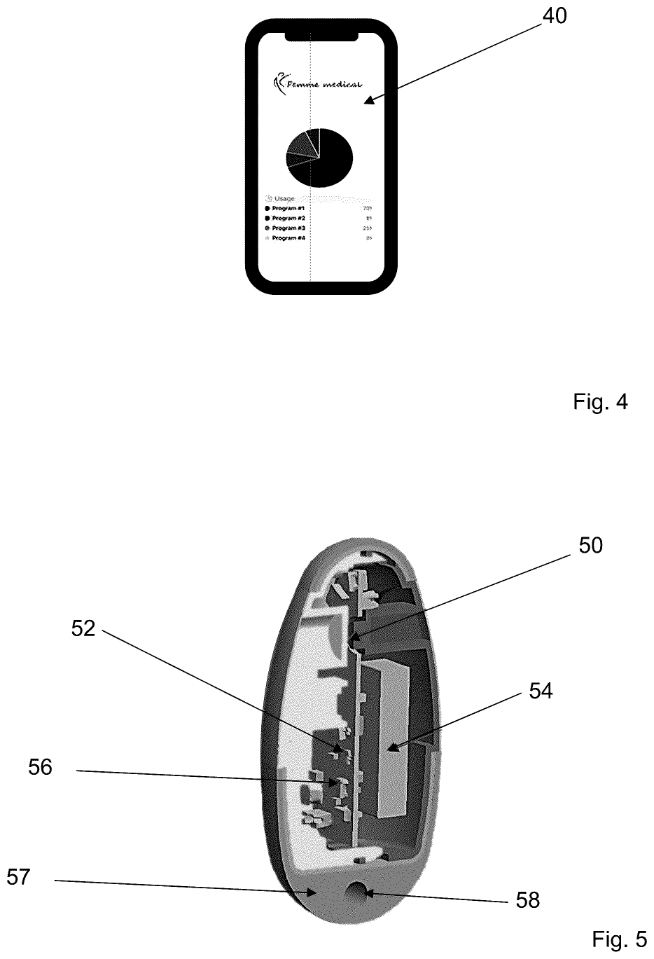

FIG. 4: An exemplary image of a user interface on an external computing device, in accordance with some embodiments.

FIG. 5: A cut-away view of a schematic illustration of an intravaginal device, in accordance with some embodiments.

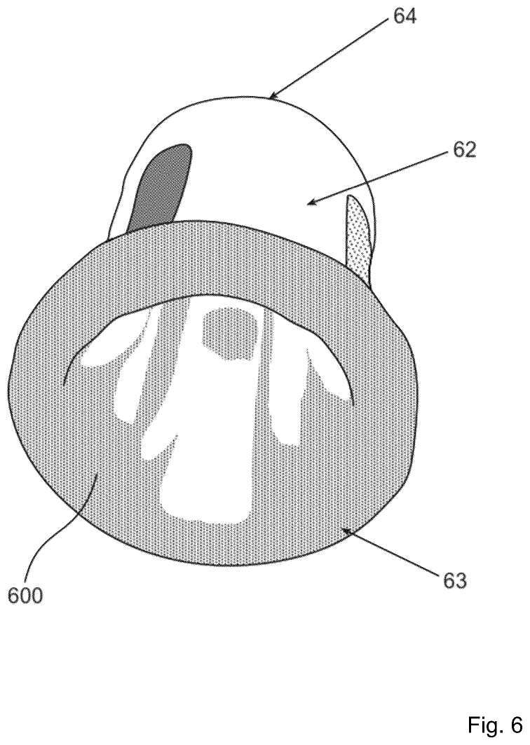

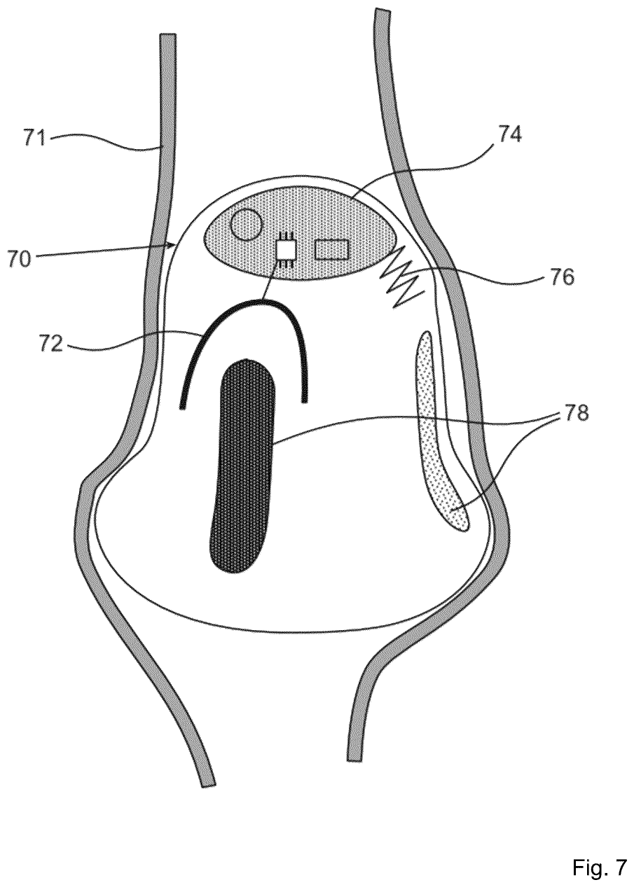

FIG. 6: A perspective view of a schematic illustration of an intravaginal device, in accordance with some embodiments.

FIG. 7: A schematic illustration of an intravaginal device positioned within the cervix, in accordance with some embodiments.

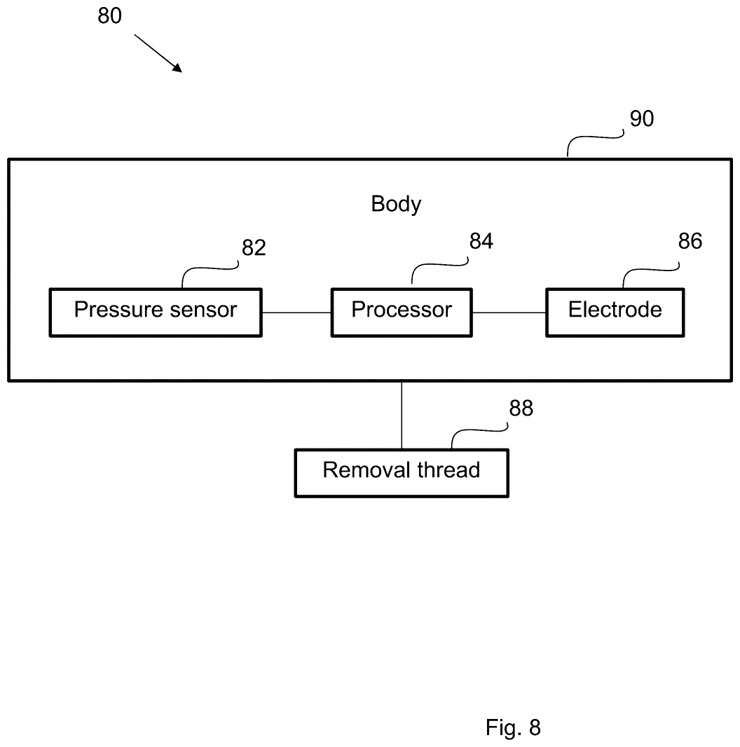

FIG. 8: A block diagram of an intravaginal device, in accordance with some embodiments.

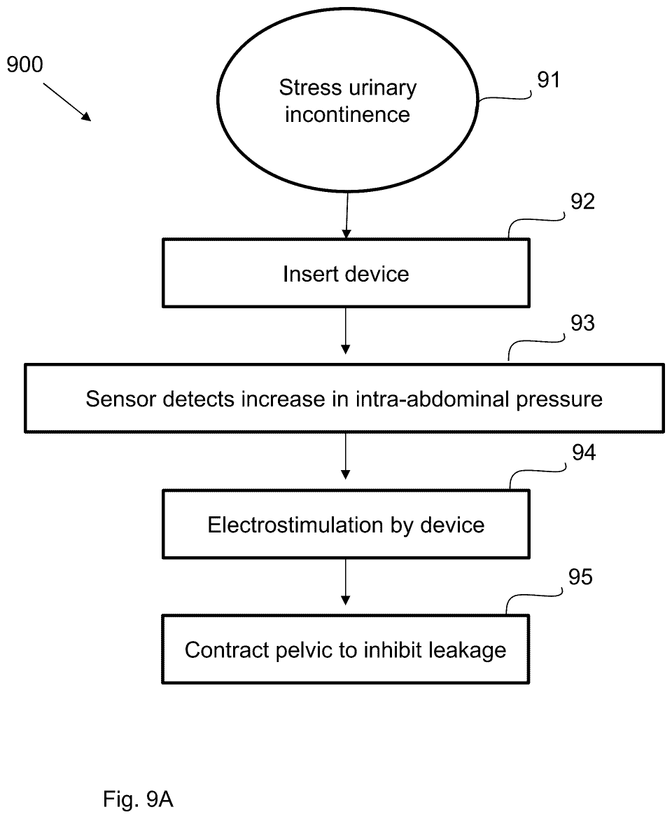

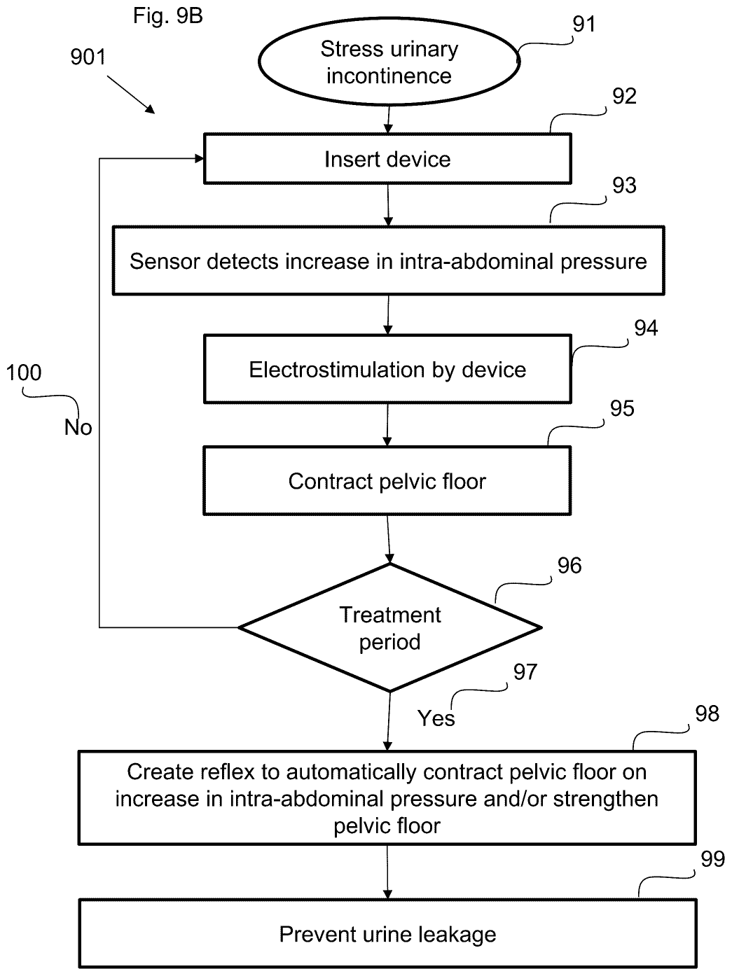

FIG. 9A: A flow chart for a method of treatment and/or prevention of stress urinary incontinence, in accordance with some embodiments.

FIG. 9B: A flow chart for a long-term method of treatment and/or prevention of stress urinary incontinence, in accordance with some embodiments.

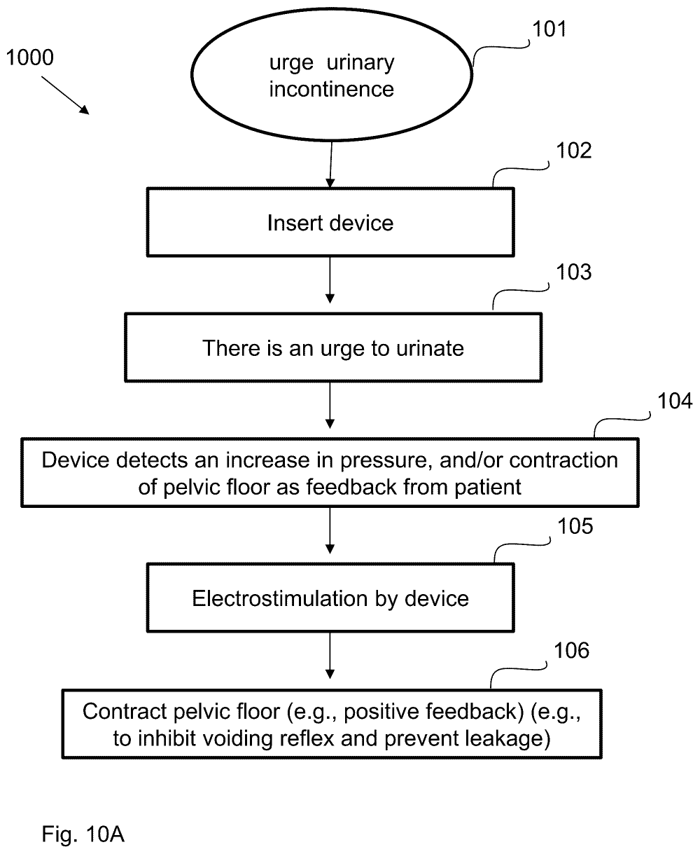

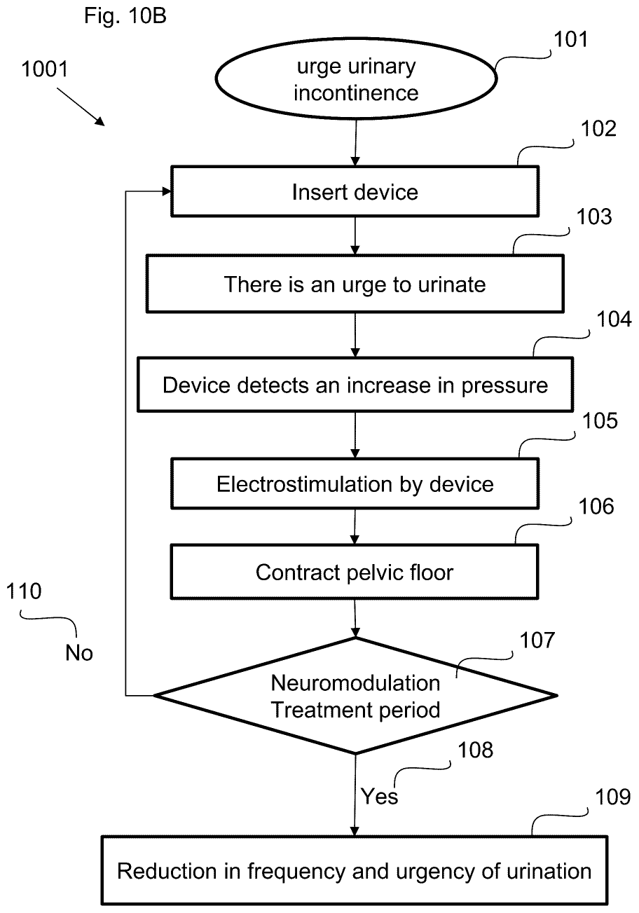

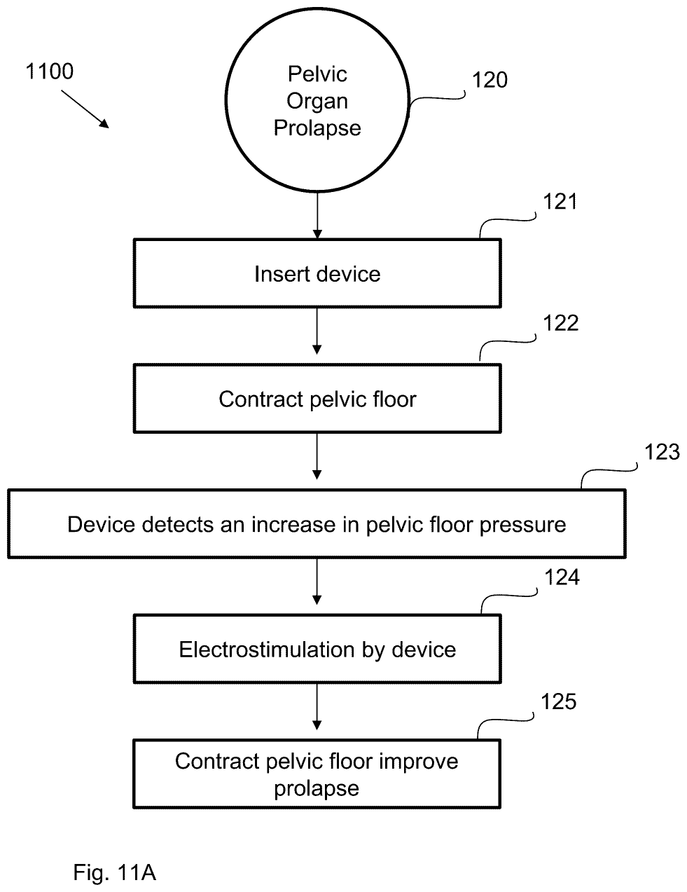

FIG. 10A: A flow chart for a method of treatment and/or prevention of urge urinary incontinence, in accordance with some embodiments.

FIG. 10B: A flow chart for a long-term method of treatment and/or prevention of urge urinary incontinence, in accordance with some embodiments.

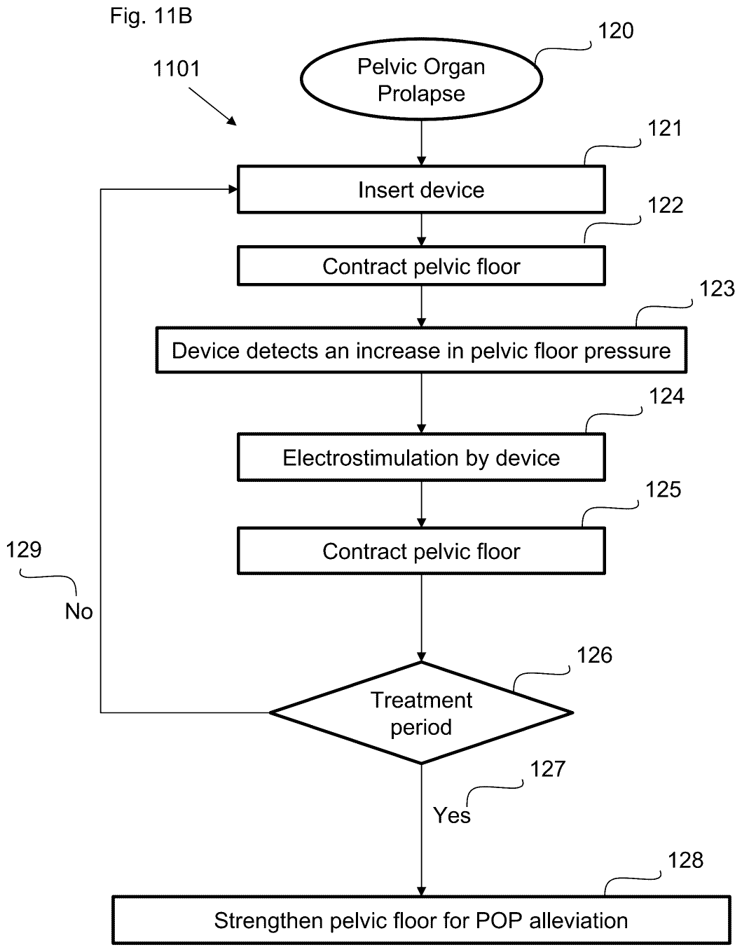

FIG. 11A: A flow chart for a method of treatment and/or prevention of pelvic organ prolapse, in accordance with some embodiments.

FIG. 11B: A flow chart for a method of treatment and/or prevention of pelvic organ prolapse, in accordance with some embodiments.

### DETAILED DESCRIPTION OF THE INVENTION

The present invention in some embodiments thereof relates to a device for the prevention and/or treatment of incontinence, and more particularly, but not exclusively, in women.

Unless otherwise defined, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### Overview

Some embodiments relate to a system and method for the prevention and/or treatment of incontinence. According to some embodiments, the incontinence may be urinary and/or fecal incontinence. According to some embodiments, the user may be biologically female.

in some embodiments, the present invention, in some embodiments thereof, combines behavioral therapy, mechanical support, and/or electrical stimulation. Further advantageously, some embodiments relate to an electro-mechanical device that includes a sensor. Optionally, the sensor may measure indicators of urinary related activity. For example, the sensor may measure intra-abdominal pressure, pelvic floor contractions and/or electromyographic activity (e.g., of the pelvic floor). According to some embodiments, the device may apply one or more electric signals. For example, the signals may stimulate muscles to support the bladder neck and/or to close the bladder neck. The signals may be initiated, stopped and/or adjusted in response to the indicators of urinary related activity. For example, in response to an increase in intra-abdominal pressure rises the device may stimulate muscles to support the bladder neck. According to some embodiments, the device may advantageously have various sizes (e.g., with, height, etc.), different coverings and/or various projections.

In some embodiments, the shape may be adapted and/or adaptable, for example, to keep the device in the vagina and/or to ensure good contact and/or to prevent descent. Optionally, a length and/or width of a device may be adapted and/or a protrusion from the device may be adaptable. For example, the device may have an interchangeable skin. Optionally, skin may be thicker and/or thinner and/or include a protrusion e.g., to fit the width of a vagina. Optionally, the skin may include a longitudinal extension e.g., to fit a length of a vagina. Optionally, the skin may include a lateral extending rim.

According to some embodiments, the device may be inserted intravaginally and/or anally. According to some embodiments, the device may be in intimate contact with the vaginal and/or rectal mucosa and/or wall. According to some embodiments, the device may be non-penetrating. According to some embodiments, the device may provide electrostimulation. According to some embodiments, the electrostimulation may be to the pelvic floor. According to some embodiments, the electrostimulation may be to one or more of a S1-S4 dermatome, vaginal wall, rectal wall, and/or perineum.

According to some embodiments, the device may include a body comprising one or more electrodes, one or more pressure sensors, and a processor. According to some embodiments, the one or more electrodes may be in intimate contact with the vaginal and/or rectal mucosa and/or wall.

According to some embodiments, the device is wearable without fear of it being dislodged and/or affecting the users' daily activities. According to some embodiments, the device may be easily inserted by the user. According to some embodiments, the device may have a rounded tip to facilitate insertion. According to some embodiments, the device may be easily removed by the user. According to some embodiments, the device may include a removal thread, loop, and/or knob. According to some embodiments, the removal thread, loop, and/or knob may be integral to and/or attached to the body of the device.

According to some embodiments, the body may be elastic. According to some embodiments, the body may be is reversibly deformed by an increase in the abdominal pressure. According to some embodiments, reversibly elastic means that the anteroposterior diameter may be reduced by at least 5% when the abdominal pressure increase by at least 0.01 bar and/or the diameter may be reduced by at least 1% by increase of pressure of at least 0.01 bar.

According to some embodiments, the body may support the vagina wall at the level of the bladder neck above the pubis. According to some embodiments, the body may include one or more lateral projections and/or a rim at the caudal part for supporting the bladder neck. According to some embodiments, the cranial segment of the body may include an axially compressible region to accommodate various lengths of the vagina and/or to position the caudal rim above the pubis. Alternatively or additionally, a length and/or width of the device and/or rim thereof may be adjusted. For example, by exchanging interchangeable skins.

According to some embodiments, the shape of the body may be conical and/or egg shaped and/or shaped as an elongated egg. Optionally, the device includes one or mor protrusion (e.g., protruding rings). For example, the protrusions may be on a caudal portion of the device. Optionally, the caudal side of the device may be wider than the cranial side. Optionally, there may be a hole in the caudal side and/or below the protrusion. For example, a removal thread may be connected to the hole. According to some embodiments, the body may have a rounded tip to facilitate insertion. According to some embodiments, the body may include a central passage.

According to some embodiments, the body may include one or more additional sensors. According to some embodiments, the one or more additional sensors may be selected from the group including: accelerometer, position, tilt, photoplethysmography, bioimpedance, ultrasonographic electromyography, etc.

According to some embodiments, the one or more pressure sensors may detect intra-abdominal pressure. According to some embodiments, the pressure sensor may be from a group including strain gauge, piezoelectric crystals, piezoelectric film, etc.

According to some embodiments, the measured pressure may be passed on to the processor. According to some embodiments, the processor may activate one or more electrodes to provide electrostimulation in response to the detected intra-abdominal pressure. According to some embodiments, the processor may be programmed to generate electrostimulation when an increase in abdominal pressure that precede an imminent urine or fecal leak is detected by a pressure sensor. According to some embodiments, the processor may be programmed to generate electrostimulation when a signal from the pressure sensors that precedes an imminent urine or fecal leak is detected.

According to some embodiments, the one or more pressure sensors may detect an increase in intra-abdominal pressure thereby inducing electrostimulation of the pelvic floor.

According to some embodiments, the body may include a battery. Optionally, the battery may be rechargeable.

According to some embodiments, the body may include a wireless communication module. Optionally, the wireless communication module may include an antenna, a blue tooth connection, a wi-fi connection, etc. According to some embodiments, the removal thread may include a wi-fi transmitter and/or an antenna for wireless communication.

According to some embodiments, the device may be in wireless communication with a user interface, data storage module and/or cloud interface and/or data storage. According to some embodiments, the device may be activated and/or setup and/or controlled through the user interface. According to some embodiments, the user interface may be an application on an external computing device (App). According to some embodiments, the external computing device may be a cellular phone, tablet, phablet, laptop, desktop, smartwatch, smart glasses, VR device, etc. According to some embodiments, sensor data, sensor recordings, stimulation parameters and timing may be transmitted wirelessly to an external computing device.

According to some embodiments, the App may facilitate wireless activation, control, and programing of the device, e.g., the cutoff values for the sensor inputs that induce an electrostimulation session. According to some embodiments, the App may facilitate wireless activation control and/or programing one or more parameters, such as, but not limited to: voltage, current intensity, shape and duration of stimulation pulses, duty cycle, modulation, session duration, pattern etc. According to some embodiments, the user may introduce comments, notifications, etc. at different times.

According to some embodiments, electrostimulation may be collected and/or stored by the processor, the App and/or a cloud connected thereto. According to some embodiments, cumulative electrostimulation data and/or cumulative sensor data and/or changes and/or trends in the data with time may be analyzed, stored, and presented to the user, e.g., in a graphical manner. In a particular embodiment, the data may be sent to other devices wirelessly.

In some embodiments, the external computer includes a user interface configured to collect feedback from a user regarding at least one of occurrence of episodes of incontinence, severity of incontinence episodes and/or context of incontinence episodes. For example, context may include Impact on quality of life: For example, impact on quality of life may include embarrassment, isolation, and/or depression. For example, impact on quality of life may difficulties in participating in activities, such as exercise, work, and social gatherings. Additionally or alternatively, quality of life context may include stigma: and/or fear being judged or ridiculed. Additionally or alternatively, quality of life context may include financial burden, for example the cost of incontinence pads, diapers, and/or other medical supplies can add up quickly. Costs may also include costs for doctor's visits, tests, and/or treatments. Contextual factors may also include individual factors that can contribute to female incontinence, for example, age, pregnancy, childbirth, menopause, medical conditions (e.g., diabetes, neurological disorders, and multiple sclerosis), obesity, behaviors (e.g., smoking alcohol use), and/or family history.

In some embodiments, the external computer is configured to use these data in order to adjust the stimulation parameters. For example, the external computer may include routines to compute stimulation parameters that will better control incontinence and/or that will have fewer negative side effects based on data from sensors and/or feedback from the user and/or other data (for example data from other users and/or data from medical personnel and/or data from medical databases). In some embodiments, the external computer and the app. is configured to present to a user data such as pelvic floor strength, improvement with time and/or improvement regarding incontinence and/or changes in treatment over time. Optionally, external computer is configured to use the data from sensors, the user, multiple users and/or other sources for training a machine learning routine. In some embodiments, a machine learning routine will include a vector machine and/or reinforcement learning.

According to some embodiments, the processor may be programmable to generate electrostimulation signals in case of an increase in abdominal pressure, or a steep increase in abdominal pressure that may lead to urine or fecal leakage.

According to some embodiments, the processor may be programmable to generate an electrostimulation signal in case of signals from the sensors that precede an imminent urine or fecal leak.

According to some embodiments, an artificial intelligence (AI) module, such as convoluted neuronal networks (CNN), vector machine, machine learning, etc. may be used for analyzing the sensors data and/or learning and/or determining if there is a high chance of an imminent urine or fecal leak. According to some embodiments, the AI module may instruct the processor to stimulate the pelvic floor, e.g., if there a high chance of an imminent urine or fecal leak is determined.

According to some embodiments, the device may be calibrated for each user. According to some embodiments, calibration may include determining the cutoff values of the sensor data that precede an imminent urine or fecal leak an order to trigger pelvic floor electrostimulation. According to some embodiments, calibration may include determining electrostimulation required to prevent and/or reduce an imminent urine or fecal leak. According to some embodiments, calibration may include determining a treat program to reduce and/or prevent urine or fecal leaks.

According to some embodiments, the device may be used for neuromodulation. According to some embodiments, the neuromodulation may include causing reflex contraction of the pelvic floor in case of an increase in abdominal pressure or other body situations that may lead to a urine and/or fecal leak. According to some embodiments, the device may be used for neuromodulation to inhibit a strong desire to urinate or to defecate. According to some embodiments, the device may be used for neuromodulation to initiate voiding and/or initiate defecation.

According to some embodiments, the device may be used to strengthen the pelvic floor. According to some embodiments, the device may be used to treat mild to moderate pelvic prolapse by strengthening the pelvic floor.

According to some embodiments, the device may exercise a users' muscles (e.g., pelvic floor, urethral sphincter, etc.). According to some embodiments, the device may strengthen a users' muscles over time. According to some embodiments, the device may thereby reduce and/or prevent urine and/or fecal leakage. According to some embodiments, the device may promote habituating proper response to urine and/or feces.

Some embodiments relate to a method for non-invasive electrostimulation of the pelvic floor including: sensing a condition that can lead to urinary or fecal incontinence; electrostimulation of the pelvic floor muscles by contacting electrodes on a body of the device on an S1-S4 dermatome or vaginal or rectal wall; and preventing urinary or fecal incontinence or inhibiting the desire to void or defecate.

Some embodiments relate to a system for stimulating the pelvic floor by an electrical current comprising one or more electrodes in intimate contact with the vaginal mucosa, rectal mucosa, the perineum, and/or a S1-S4 dermatome; and at least one sensor for detecting intra-abdominal pressure, wherein detection of increasing intra-abdominal pressure by a sensor induces electrostimulation of the pelvic floor to prevent urinary or fecal incontinence.

### Specific Embodiments

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Reference is Now Made to the Figures

FIG. 1 is a schematic illustration of a side view of an intravaginal device, in accordance with some embodiments. For example, the device may include a body 11 with a rounded cranial tip 14 and/or a rounded caudal tip 15. Optionally, the body 11 is fully or partially covered with an elastic skin 10. For example, skin 10 may cover a cranial portion of body 11 and/or leave exposed electrodes 12a and 12b on an outer surface of the body 11. For example, electrode 12a may be charged with an opposite charge from electrode 12b. In some embodiments, skin 10 may include one or more projections 16, for example, projections 16 may surrounding and/or project laterally from a caudal portion of the skin 10. Optionally, the projections 16 are configured to support a bladder neck of a user of the device. In some embodiments, skin 10 may be removed and/or replaced, for example, with a different skin to fit a different shaped vagina. Optionally, a removal thread 18 is attached to a caudal portion of the body 11.

In some embodiments, the electro-stimulation may range from 5 to 15 volts and/or 1 to 5 volts and/or 0.1 to 1 volt and/or 15 to 100 volts.

In some embodiments, the electro-stimulation current may range from 1 to 5 milliamps and/or from 0.1 to 1 milliamp, and/or from 5 to 20 milliamp and/or from 20 to 100 milliamp.

In some embodiments, the electro-stimulation current may range from range from 10- to 50 hertz and/or between 1 to 10 hertz and/or 50 to 250 hertz and/or 250 to 1000 Hz and/or 1000 to 20000 Hz.

According to some embodiments, the body may be elastic. Optionally, the body 11 and/or skin 10 may be is reversibly deformed by an increase in the abdominal pressure e.g., when the abdominal pressure increases by at least 0.01 bar, the anteroposterior diameter of the body may be reduced by at least 1%, and/or at least 5%, and/or by at least 10%.

FIG. 2 is a schematic illustration of a side view of an intravaginal device, in accordance with some embodiments. For example, the device may include a body 21. In some embodiments, the body 21 may have an ovoid shape. The body 21 optionally includes a rounded cranial tip 28 and/or a rounded caudal tip 29. Optionally, the body 21 is fully or partially covered with an elastic skin 20. For example, skin 20 may cover a cranial portion of body 21 and/or leave exposed electrodes 24 on an outer surface of the body 21. In some embodiments, the skin 20 may include one or more protrusions 22. Optionally, a removal thread 26 is connected to a caudal portion of the body. One or more electrodes 24 and one or more electrodes. Optionally, the one or more pressure sensors may be located on the surface of the body and/or under the skin 20. Optionally, the one or more pressure sensors may be located under the skin of the body, close to an outer surface thereof. Optionally, the removal thread may include a wi-fi transmitter and/or antenna.

FIG. 3 shows side views of multiple covers on an intravaginal device, in accordance with some embodiments. For example, a device 30 may include different interchangeable elastic skins 35 for covering the device. Optionally, different regions of the device 30 may include different types of skin/surface coverings. Preferably, the skin/surface covering may be anti-allergic and/or non-toxic and/or biocompatible. For example, the skin 35 may be made of rubber and/or elastomer and/or plastic and/or silicon.

According to some embodiments, the skins 35 may be different sizes to fit different vaginas and/or rectums. Optionally, the device 30 and/or skin 35 may have a width ranging between about 1 cm to about 2 cm, and/or 2 cm to about 3 cm, and/or about 3 cm to about 5 cm, etc. Optionally, the device 30 and/or the skin 35 may have a length ranging between about 2 cm to about 3 cm, and/or about 3 cm to about 5 cm, and/or between about 5 cm to about 8 cm, etc.

According to some embodiments, the device 30 and/or skin 35 may include a longitudinal projection 34. Optionally, different interchangeable skins may have different sized projections 34. Optionally, the longitudinal projection 34 is axially compressible region and/or of various lengths to accommodate various lengths of the vagina, etc. and/or to position the caudal rim above the pubis. Optionally, the length of the axially compressible region may range between about 0 cm to about 1 cm, and/or between about 1 cm to about 2 cm, and/or between about 2 cm to about 3 cm, and/or between about 3 cm to about 5 cm.

According to some embodiments, the device 30 and/or the skin 35 may include one or more lateral projections. For example, a lateral projection may include a rim 32. For example, a rim 32 may be positioned on a caudal portion of the device 30 and/or may be part of an exchangeable skin 35. For example, the rim 32 may be configured to support the bladder neck. Optionally, the lateral projections may include protrusions of various sizes and/or diameters and/or distances apart. Optionally, a lateral protrusion may include a bulge on the side of the device. Optionally, the one or more lateral projections may assist in maintaining the device 30 in place. Optionally, the one or more lateral projections may prevent descent of bladder neck. Optionally, a lateral projection may improve contact of a sensor and/or electrode with tissue. For example, the protrusion may be opposite the sensor and/or electrode thereby pushing the side sensor and/or electrode against the tissue. Optionally, a protrusion and/or projection may be elastic and/or have a shape memory (e.g., shape memory foam). For example, an elasticity facilitate insertion and/or removal of the device while keeping the device in place while in use.

FIG. 4 is an exemplary image of a user interface on an external computing device, in accordance with some embodiments. For example, the inserted device may include a wireless communication module and/or be in wireless communication with a user interface 40, e.g., App on an external computing device. According to some embodiments, the external computing device may be a cellular phone, tablet, phablet, laptop, desktop, smartwatch, smart glasses, VR device, etc. According to some embodiments, sensor data, sensor recordings, stimulation parameters and timing may be transmitted wirelessly to an external computing device. According to some embodiments, the device may be activated and/or settings and/or setup and/or may be controlled and/or adjusted according to wireless signals of the external computing device and/or under control of the user interface. Alternatively or additionally, the app. on the external device may include instructions to adjust the settings of the inserted device automatically in response to the data received from the inserted device. The automatic adjustments are optionally adjustable and/or are adjusted using the user interface of the external computing device.

According to some embodiments, the App may facilitate wireless activation, control, and programing of the device, e.g., the cutoff values for the sensor inputs that induce an electrostimulation session. According to some embodiments, the App may facilitate wireless activation control and/or programing one or more parameters, such as, but not limited to: voltage, current intensity, shape and duration of stimulation pulses, duty cycle, modulation, session duration, pattern etc. According to some embodiments, the user may introduce comments, notifications, etc. at different times.

According to some embodiments, electrostimulation may be collected and/or stored by the processor, the App and/or a cloud connected thereto. According to some embodiments, cumulative electrostimulation data and/or cumulative sensor data and/or changes and/or trends in the data with time may be analyzed, stored, and presented to the user, e.g., in a graphical manner. In a particular embodiment, the data may be sent to other devices wirelessly.

FIG. 5 is a cut-away view of a schematic illustration of an intravaginal device, in accordance with some embodiments. In some embodiments, the device has an ovoid body 57. For example, the body 57 of the device may include a hole 58 for attachments, for example a removal string may be threaded through the hole 58. Optionally, hole 58 may be located on a caudal portion of the body 57. In some embodiments, battery 54, which may optionally be rechargeable, one or more pressure sensors 50, one or more electrodes 52 and a processor 56 may be mounted in an internal space within the body 57.

FIG. 6 is a perspective view of a schematic illustration of a body 62 of an intravaginal device, in accordance with some embodiments. According to some embodiments, the body 62 may include a central passage 60 and/or a lateral projection 63. For example, the projection 63 may surround a caudal portion of the body 62 and/or may be configured to support a bladder neck of the user.

In a particular embodiment, the cranial segment 64 of the body 62 is may include an axially compressible region (2) to accommodate various lengths of the vagina and to position the projection 63 (e.g., the caudal rim) above the pubis. In a particular embodiment, the body 62 may include a removal thread. In a particular embodiment, the removal thread comprises an antenna for wireless communication.

FIG. 7 is a schematic illustration of an intravaginal device positioned within the cervix, in accordance with some embodiments. For example, device body 70 may be inserted into the cervix, where the body of the device may be in contact with the vaginal mucosa and/or walls 71. An optional pressure sensor 72 on the device may detect intra-abdominal pressure. The data from the pressure sensor 72 may be analyzed by a processor 74 and electrostimulation may be provided by one or more electrodes 78 to prevent leakage of urine or feces. Optionally, a wireless communication module and/or antenna 76 supplies connection to an external device.

FIG. 8 is a block diagram of an intravaginal device 80, in accordance with some embodiments. For example, the device may include a body 90 with a rounded tip, an axially compressible region, one or more lateral projections and a removal thread 88, which may include an antenna. The body may include one or more pressure sensors 82. and one or more electrodes 86, a processor 84 and/or a battery (not shown).

FIG. 9A is a flow chart for a method 900 of treatment and/or prevention of stress urinary incontinence, in accordance with some embodiments. Alternatively or additionally, the method may be used for other urinary difficulties. For example, in method 900 a device is inserted 92 intravaginally in a user with stress urinary incontinence 91. An increase in abdominal pressure beyond a specific threshold is detected 93 by a sensor within the device. The device sends electrical stimulation 94 to the pelvic floor, causing it to contract 95. For example, the contraction 95 may create pressure of the pelvic floor and/or the urethra against the rim of the device. Optionally, the contraction 95 and/or pressures inhibits and/or prevents leakage.

FIG. 9B illustrates a long-term method 901 of using the device in accordance with some embodiments. According to some embodiments, the device may be used for a specific treatment period 96, e.g., 1 week, two weeks, three weeks, a month, a month and a half, two months, etc. If at the end of the treatment period contraction has helped 97 to prevent urine leakage effectively alleviating stress incontinence, strengthening the pelvic floor and creating a reflex 98 where the pelvic floor contracts automatically when there is an increase in intra-abdominal pressure, preventing urine leakage 99, then there is no need to continue using the device. If the user still suffers 100 from stress urinary incontinence at the end of the treatment period, then second treatment period may be initiated. Optionally, one or more parameters of the electrostimulation may be altered in the second treatment period, e.g., voltage, current intensity, shape and duration of stimulation pulses, duty cycle, modulation, session duration, pattern etc.

FIG. 10A is a flow chart for a method of treatment and/or prevention of urge urinary incontinence (UUI) 101, in accordance with some embodiments. Alternatively or additionally, the method may be used for other urinary difficulties. For example, in method 1000, a device is inserted 102 intravaginally in a user with urge urinary incontinence. The user may experience a sudden urge to urinate 103. The device may detect 104 an increase in pressure and/or a contraction of the pelvic floor, which may occur reflectively or intentionally. For example, the pressure may be in the abdominal pressure and/or of pressure on the pelvic floor. Optionally, the device may send electrical stimulation 105 (e.g., to the pelvic floor). For example, the stimulation may be in response to the increase in pressure and/or contraction of the pelvic floor. In some embodiments, the stimulation may cause the abdomen and/or the pelvic floor to contract 106 and/or suppress the urge to urinate and/or leakage.

FIG. 10B is a flow chart for a long-term method 1001 of treatment and/or prevention of urge urinary incontinence (UUI) 101, in accordance with some embodiments. According to some embodiments, the device may be used for a specific treatment period, e.g., at least 1 week, two weeks, three weeks, a month, a month and a half, two months, etc. In some cases, the end of the treatment period the device helps 108 to train the nerves and muscles of the pelvic floor, leading to a greater bladder filling volume before feeling the need to urinate (neuromodulation 107). This may result in reduction 109 in the frequency and/or urgency of urination and strengthening of the pelvic floor. If the user still suffers 110 from urge urinary incontinence at the end of the treatment period, then second treatment period may be initiated. Optionally, one or more parameters of the electrostimulation may be altered in the second treatment period, e.g., voltage, current intensity, shape and duration of stimulation pulses, duty cycle, modulation, session duration, pattern etc.

FIG. 11A is a flow chart for a method of treatment and/or prevention of pelvic organ prolapse (POP) 102, in accordance with some embodiments. For example, in method 1100, a user may be interested in strengthening the pelvic floor. A device is inserted 121 intravaginally in a user. The user contracts the pelvic floor on their own 122. The device detects 123 an increase in pelvic floor pressure. The device sends electrical stimulation 124 to the pelvic floor, causing it to contract 125. In some embodiments, the contraction 125 may improve POP. Alternatively or additionally, the method may be used for other urinary difficulties.

FIG. 11B is a flow chart for a long-term method 1101 of treatment and/or prevention of pelvic organ prolapse (POP) 120, in accordance with some embodiments. According to some embodiments, the device may be used for a specific treatment period 126, e.g., 1 week, two weeks, three weeks, a month, a month and a half, two months, etc. If at the end of the treatment period the pelvic floor has been strengthened 127 and the POP has been alleviated 128, then there is no need to continue using the device. If the user still suffers 129 from pelvic organ prolapse at the end of the treatment period, then second treatment period may be initiated. Optionally, one or more parameters of the electrostimulation may be altered in the second treatment period, e.g., voltage, current intensity, shape and duration of stimulation pulses, duty cycle, modulation, session duration, pattern etc.

It is expected that during the life of a patent maturing from this application many relevant building technologies, artificial intelligence methodologies, computer user interfaces, image capture devices will be developed and the scope of the terms for design elements, analysis routines, user devices is intended to include all such new technologies a priori.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) and/or a mesh network (meshnet, emesh) and/or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks might be expected to use completely different methods, e.g., making use of expert knowledge and/or the pattern recognition capabilities of the human brain, which would be vastly more efficient than manually going through the steps of the methods described herein.

Data and/or program code may be accessed and/or shared over a network, for example the Internet. For example, data may be shared and/or accessed using a social network. A processor may include remote processing capabilities for example available over a network (e.g., the Internet). For example, resources may be accessed via cloud computing. The term “cloud computing” refers to the use of computational resources that are available remotely over a public network, such as the internet, and that may be provided for example at a low cost and/or on an hourly basis. Any virtual or physical computer that is in electronic communication with such a public network could potentially be available as a computational resource. To provide computational resources via the cloud network on a secure basis, computers that access the cloud network may employ standard security encryption protocols such as SSL and PGP, which are well known in the industry.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An electrostimulation device for stimulating the pelvic floor comprising:
an electrode configured for intimate contact with vaginal or rectal mucosa;
a sensor for detecting urinary related activity; and
a processor configured to receive signals from said sensor, and in response to detection of urinary related activity by said sensor, induce electrostimulation of a pelvic floor by said electrode.

2. The device of claim 1, wherein said urinary related activity includes at least one of increase of intra-abdominal pressure, pelvic floor contraction, and electromyographic activity of the pelvic floor.

3. The device of claim 1, wherein the sensor includes a pressure sensor and said urinary related activity is an increase in intra-abdominal pressure.

4. The device of claim 3, wherein the pressure sensor is selected from a group consisting of: strain gauge, piezoelectric crystal, piezoelectric film, balloon and any combination thereof.

5. The device of claim 1, wherein the processor and electrode are configured to respond to said sensor sensing increased activity of pelvic floor muscles by stimulating the pelvic floor muscles with positive feedback.

6. The device of claim 5, wherein said positive feedback suppresses a voiding reflex.

7. The device of claim 1, wherein a length of the device is adaptable to fit the length of a vagina.

8. The device of claim 1, wherein at least one of a diameter and a lateral protrusion from the device is adaptable to fit a width of a vagina.

9. The device of claim 1, further comprising a rim.

10. The device of claim 9, where the rim is flexible.

11. The device of claim 10 wherein flexibility of the rim facilitates insertion or removal of the device.

12. The device of claim 1, wherein the device is configured to strengthen the pelvic floor and treat mild to moderate pelvic floor prolapse.

13. The device of claim 1, further including a wireless communication module for sending data to an external computer and receiving signals from the external computer to adjust stimulation parameters.

14. The device of claim 1, further comprising:
a body is configured to support a vagina wall at a level of a bladder neck above a pubis.

15. The device of claim 1, further comprising:
an elastic body which is reversibly deformed by an increase in intra-abdominal pressure.

16. The device of claim 15, wherein the reversible deformation is a reversible reduction of an anteroposterior diameter of the body by at least 5% when the intra-abdominal pressure increases by at least 1/10 bar.

17. The device of claim 14, wherein the body has an axially compressible region.

18. The device of claim 14, wherein the body includes a lateral projection.

19. The device of claim 18, wherein the lateral projection includes a rim around the body of the device.

20. The device of claim 18, further comprising a lateral projection configured to support a bladder neck.

21. The device of claim 1, further comprising a removal thread hanging caudally from the device and wherein the removal thread comprises an antenna for wireless communication.

22. The device of claim 1, further comprising a wireless communication module.

23. The device of claim 22, wherein sensors recordings, stimulation parameters and timing are transmitted wirelessly to an external computing device, wherein the external computing device comprises a user interface for controlling the device.

24. The device of claim 1, further comprising one or more additional sensors, wherein the one or more additional sensors are selected from a group consisting of: accelerometer, position, tilt, photoplethysmography, bioimpedance, ultrasonographic electromyography, and any combination thereof.

25. The device of claim 1, wherein said processor is configured to administer said electrostimulation in a manner that strengthens pelvic floor muscles.

26. The device of claim 1, wherein said processor is configured to administer said electrostimulation in a manner that produces neuromodulation.

27. The device of claim 26, wherein the neuromodulation includes reflex contraction of the pelvic floor on increase in intra-abdominal pressure.

28. The device of claim 26, wherein the neuromodulation includes inhibition of a desire to urinate or to defecate.

29. The device of claim 1, wherein the electrostimulation causes neuromodulation which initiates voiding or defecation.

30. A method for non-invasive electrostimulation of pelvic floor muscles comprising:
sensing a urinary related activity;
electrostimulating the pelvic floor muscles by at least one electrode in contact with a vaginal or rectal mucosa in response to said sensing to contract the pelvic floor muscles in response to said electrostimulation.

31. The method of claim 30, further comprising:
Repeating said sensing and electrostimulation to create a reflex to automatically contract the pelvic floor on increase of intra-abdominal pressure.

32. The method of claim 30, wherein said electrostimulating reduces at least one of frequency and urgency of urination.

33. The method of claim 30, further comprising:
Repeating said sensing and electrostimulation to strengthen pelvic floor muscles.

34. The method of claim 30, further comprising:
controlling the electrostimulation by a processor is connected to at least one pressure sensor and at least one electrode.

35. The method of claim 34, wherein the processor further comprises an artificial intelligence (AI) module.

36. The method of claim 35, further comprising:
determining with the AI module when there is a high chance of an imminent urine or fecal leak.

37. The method of claim 36, further comprising:
instructing the at least one electrode to provide electrostimulation is response to said determining.

38. A system for electrostimulation of pelvic floor muscles comprising:
an insertable device for insertion into a vagina including
an electrode configured for intimate contact with vaginal or rectal mucosa,
a sensor for detecting urinary related activity and
a wireless communication module,
a processor configured to receive signals from said sensor, and in response to detection of increasing intra-abdominal pressure by said pressure sensor, induce electrostimulation of the pelvic floor by said electrode; and
a user interface linked wirelessly to said processor, wherein the user interface is an application on an external computer.

39. The system of claim 38 wherein said urinary related activity includes at least one of an increase of intra-abdominal pressure, pelvic floor contraction, and electromyographic activity of the pelvic floor.

40. The system of claim 38, wherein said wireless communication module is configured for sending data to said external computer and receiving signals from the external computer to adjust stimulation parameters.

41. The system of claim 38, wherein said external computer includes a user interface configured to collect feedback from a user regarding at least one of occurrence, severity of incontinence episodes and context of incontinence episodes.

42. The system of claim 41, wherein said external computer is configured to use these data for adjusting parameters of the stimulation.

43. The system of claim 41, wherein the external computer is configured to present to a user data on at least one of pelvic floor strength, improvement with time and improvement regarding incontinence treatment.

44. The system of claim 42, wherein the external computer is configured to use data from multiple users is for said adjusting.

45. The system of claim 44, wherein the external computer is configured to use said data from multiple users for training a machine learning routine.

46. The system of claim 45 wherein includes at least one of a vector machine and reinforcement learning.

47. The system of claim 38, wherein the user interface is configured to facilitate wireless activation, control, and/or programing of one or more parameters of the insertable device.

48. The system of claim, 47, wherein the one or more parameters are selected from the group consisting of: cutoff values for the sensor signals that induce electrostimulation, voltage, current intensity, shape and duration of stimulation pulses, duty cycle, modulation, session duration, patterns, and any combination thereof.

49. The system of claim 38, wherein the external computer is a cellular phone, tablet, phablet, laptop, desktop, smartwatch, smart glasses, or VR device.

50. The system of claim 49, wherein the external computer is configured to analyze, store, and present to user cumulative electrostimulation data, cumulative sensor data, changes, and trends in the data in a graphical manner.
